SEQUENCE LISTING (1 sequence)

SEQ ID NO: 1 - protein, 1771 aa, Homo sapiens
MSVLISQSVINYVEEENIPALKALLEKCKDVDERNECGQTPLMIAAEQGNLEIVKELIKNGANCNLEDLDNWTALISASKEGHVHIVEELLKCGVNLEHRDMGGWTALMWACYKGRTDVVELLLSHGANPSVTGLYSVYPIIWAAGRGHADIVHLLLQNGAKVNCSDKYGTTPLVWAARKGHLECVKHLLAMGADVDQEGANSMTALIVAVKGGYTQSVKEILKRNPNVNLTDKDGNTALMIASKEGHTEIVQDLLDAGTYVNIPDRSGDTVLIGAVRGGHVEIVRALLQKYADIDIRGQDNKTALYWAVEKGNATMVRDILQCNPDTEICTKDGETPLIKATKMRNIEVVELLLDKGAKVSAVDKKGDTPLHIAIRGRSRKLAELLLRNPKDGRLLYRPNKAGETPYNIDCSHQKSILTQIFGARHLSPTETDGDMLGYDLYSSALADILSEPTMQPPICVGLYAQWGSGKSFLLKKLEDEMKTFAGQQIEPLFQFSWLIVFLTLLLCGGLGLLFAFTVHPNLGIAVSLSFLALLYIFFIVIYFGGRREGESWNWAWVLSTRLARHIGYLELLLKLMFVNPPELPEQTTKALPVRFLFTDYNRLSSVGGETSLAEMIATLSDACEREFGFLATRLFRVFKTEDTQGKKKWKKTCCLPSFVIFLFIIGCIISGITLLAIFRVDPKHLTVNAVLISIASVVGLAFVLNCRTWWQVLDSLLNSQRKRLHNAASKLHKLKSEGFMKVLKCEVELMARMAKTIDSFTQNQTRLVVIIDGLDACEQDKVLQMLDTVRVLFSKGPFIAIFASDPHIIIKAINQNLNSVLRDSNINGHDYMRNIVHLPVFLNSRGLSNARKFLVTSATNGDVPCSDTTGIQEDADRRVSQNSLGEMTKLGSKTALNRRDTYRRRQMQRTITRQMSFDLTKLLVTEDWFSDISPQTMRRLLNIVSVTGRLLRANQISFNWDRLASWINLTEQWPYRTSWLILYLEETEGIPDQMTLKTIYERISKNIPTTKDVEPLLEIDGDIRNFEVFLSSRTPVLVARDVKVFLPCTVNLDPKLREIIADVRAAREQISIGGLAYPPLPLHEGPPRAPSGYSQPPSVCSSTSFNGPFAGGVVSPQPHSSYYSGMTGPQHPFYNRPFFAPYLYTPRYYPGGSQHLISRPSVKTSLPRDQNNGLEVIKEDAAEGLSSPTDSSRGSGPAPGPVVLLNSLNVDAVCEKLKQIEGLDQSMLPQYCTTIKKANINGRVLAQCNIDELKKEMNMNFGDWHLFRSTVLEMRNAESHVVPEDPRFLSESSSGPAPHGEPARRASHNELPHTELSSQTPYTLNFSFEELNTLGLDEGAPRHSNLSWQSQTRRTPSLSSLNSQDSSIEISKLTDKVQAEYRDAYREYIAQMSQLEGGPGSTTISGRSSPHSTYYMGQSSSGGSIHSNLEQEKGKDSEPKPDDGRKSFLMKRGDVIDYSSSGVSTNDASPLDPITEEDEKSDQSGSKLLPGKKSSERSSLFQTDLKLKGSGLRYQKLPSDEDESGTEESDNTPLLKDDKDRKAEGKVERVPKSPEHSAEPIRTFIKAKEYLSDALLDKKDSSDSGVRSSESSPNHSLHNEVADDSQLEKANLIELEDDSHSGKRGIPHSLSGLQDPIIARMSICSEDKKSPSECSLIASSPEENWPACQKAYNLNRTPSTVTLNNNSAPANRANQNFDEMEGIRETSQVILRPSSSPNPTTIQNENLKSMTHKRSQRSSYTRLSKDPPELHAAASSESTGFGEERESIL

(19) United States
(12) Patent Application Publication
(10) Pub. No.: US 2026/0056214 A1
(43) Pub. Date: Feb. 26, 2026

(54) DIAGNOSING AND TREATING END-STAGE RENAL DISEASE (ESRD) IN OBESE SUBJECTS

(71) Applicant: THE BOARD OF REGENTS OF THE UNIVERSITY OF OKLAHOMA, Norman, OK (US)

(72) Inventor: Jesse R. RICHARDS, Tulsa, OK (US)

(73) Assignee: THE BOARD OF REGENTS OF THE UNIVERSITY OF OKLAHOMA, Norman, OK (US)

(21) Appl. No.: 19/310,109

(22) Filed: Aug. 26, 2025

Related U.S. Application Data
(60) Provisional application No. 63/686,988, filed on Aug. 26, 2024.

(51) Int. Cl. G01N 33/68

(52) U.S. Cl. CPC G01N 33/6893; G01N 2800/347; G01N 2800/50; G01N 2800/52

(57) ABSTRACT

A method of evaluating a subject for a renal condition, wherein the subject is obese or severely obese, and treating the subject based on the evaluation, the method comprising the steps of (1) performing an assay configured to detect a non-benign Kinase D-interacting substrate of 220 kDA (KIDINS220) variant protein in a body fluid sample obtained from the subject; (2) determining that the subject has an elevated risk for end-stage renal disease (ESRD) when one or more non-benign KIDINS220 variants are present in the body fluid sample; and (3) treating the subject having the elevated risk for ESRD with a compatible kidney treatment regimen.

### CROSS REFERENCE TO RELATED APPLICATIONS

This application claims the benefit of U.S. Provisional Patent Application Ser. No. 63/686,988, filed Aug. 26, 2024, the content of which is hereby expressly incorporated herein by reference in its entirety.

### STATEMENT OF GOVERNMENT SUPPORT

Not applicable.

### REFERENCE TO A SEQUENCE LISTING

This application contains a Sequence Listing XML, which has been submitted electronically and is hereby incorporated by reference in its entirety. Said Sequence Listing XML, created on Aug. 26, 2025, is named OKLAP0031US_ST26.xml and is 3,733 bytes in size.

### BACKGROUND

End stage renal disease (ESRD) frequently leads to kidney transplantation, which involves not only a major surgical operation, but also a lifetime of medical and immunosuppressive therapy post-transplant. Currently, despite decades of research, there are no well-identified methods of predicting renal disease. Recently genetic testing for inherited renal diseases has been expanding. Existing genetic panels can be useful since apparently about 25% of general chronic kidney disease patients, and up to 65% of patients with a family history of renal disease, potentially have an underlying genetic contribution to their kidney disease. However, additional markers, especially for novel pathways, are still needed to improve risk stratification of patients who might progress to ESRD and thus who would eventually require dialysis or a kidney transplant. Such markers could also be used to screen kidney transplant donors to evaluate the appropriateness for organ transplant.

### DETAILED DESCRIPTION

The present disclosure is directed to a method of identifying subjects who are at high risk for ESRD and who therefore may need more aggressive medical treatment or who are at high risk of needing a kidney transplant. The method uses the presence of mutations in Kinase D-interacting substrate of 220 kDa (“KIDINS220”) protein (i.e., “KIDINS220 variants”) that previously have not been connected to renal disease. Using this method, patients who do not necessarily have an easily identified cause of renal failure can be prospectively assessed for the risk of renal disease progression. The method can also be integrated into existing panels to improve predictive accuracy. This technology can be used by medical personnel to screen the general population of patients at-risk for renal disease or in patients with a family history of renal disease to help risk stratify those who need more intensive risk factor modification or more expensive preventative treatments.

Before further describing various embodiments of the compositions, kits, and methods of the present disclosure in more detail by way of exemplary description, examples, and results, it is to be understood that the present disclosure is not limited in application to the details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure as defined herein. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

Where used herein, the pronoun “we” is intended to refer to all persons involved in a particular aspect of the investigation disclosed herein and as such may include non-inventor laboratory assistants and non-inventor collaborators working under the supervision of the inventor(s).

As utilized in accordance with the methods, compounds, and compositions of the present disclosure, the following terms, unless otherwise indicated, shall be understood to have the following meanings.

The term “KIDINS220 variant” refers to a human KIDINS220 protein of 1771 amino acids that comprises one or more mutations in the amino acid sequence of the wild-type KIDINS220 protein (see Table 1). Such mutations may include, in non-limiting examples, deletions or insertions of one or more amino acids, amino acid substitutions, missense mutations, nonsense mutations, and frameshift mutations. The term “benign” (as in “benign variant” or “benign mutation”) where used herein, refers to a variant that possesses substantially the same activity as the wild-type protein and is not harmful to the bearer of the variant. Where used herein, the term “benign variant mutation” may refer to a mutation in a gene that does not affect expression of the mutant gene or the activity of the protein encoded by the mutant gene. Where used herein, the term “non-benign mutation” refers to a variant or mutation in a gene or protein that negatively affects expression of the mutant gene or negatively affects the normal activity of the protein encoded by the mutant gene.

TABLE 1
KIDINS220 canonical amino acid sequence (Human)

MSVLISQSVINYVEEENIPALKALLEKCKDVDERNECGQTPLMIAAEQGNLEIVKELIKN
GANCNLEDLDNWTALISASKEGHVHIVEELLKCGVNLEHRDMGGWTALMWACYKGRTDVV
ELLLSHGANPSVTGLYSVYPIIWAAGRGHADIVHLLLQNGAKVNCSDKYGTTPLVWAARK
GHLECVKHLLAMGADVDQEGANSMTALIVAVKGGYTQSVKEILKRNPNVNLTDKDGNTAL
MIASKEGHTEIVQDLLDAGTYVNIPDRSGDTVLIGAVRGGHVEIVRALLQKYADIDIRGQ
DNKTALYWAVEKGNATMVRDILQCNPDTEICTKDGETPLIKATKMRNIEVVELLLDKGAK
VSAVDKKGDTPLHIAIRGRSRKLAELLLRNPKDGRLLYRPNKAGETPYNIDCSHQKSILT
QIFGARHLSPTETDGDMLGYDLYSSALADILSEPTMQPPICVGLYAQWGSGKSFLLKKLE
DEMKTFAGQQIEPLFQFSWLIVFLTLLLCGGLGLLFAFTVHPNLGIAVSLSFLALLYIFF
IVIYFGGRREGESWNWAWVLSTRLARHIGYLELLLKLMFVNPPELPEQTTKALPVRFLFT
DYNRLSSVGGETSLAEMIATLSDACEREFGFLATRLFRVFKTEDTQGKKKWKKTCCLPSF
VIFLFIIGCIISGITLLAIFRVDPKHLTVNAVLISIASVVGLAFVLNCRTWWQVLDSLLN
SQRKRLHNAASKLHKLKSEGFMKVLKCEVELMARMAKTIDSFTQNQTRLVVIIDGLDACE
QDKVLQMLDTVRVLFSKGPFIAIFASDPHIIIKAINQNLNSVLRDSNINGHDYMRNIVHL
PVFLNSRGLSNARKFLVTSATNGDVPCSDTTGIQEDADRRVSQNSLGEMTKLGSKTALNR
RDTYRRRQMQRTITRQMSFDLTKLLVTEDWFSDISPQTMRRLLNIVSVTGRLLRANQISF
NWDRLASWINLTEQWPYRTSWLILYLEETEGIPDQMTLKTIYERISKNIPTTKDVEPLLE
IDGDIRNFEVFLSSRTPVLVARDVKVFLPCTVNLDPKLREIIADVRAAREQISIGGLAYP
PLPLHEGPPRAPSGYSQPPSVCSSTSFNGPFAGGVVSPQPHSSYYSGMTGPQHPFYNRPF
FAPYLYTPRYYPGGSQHLISRPSVKTSLPRDQNNGLEVIKEDAAEGLSSPTDSSRGSGPA
PGPVVLLNSLNVDAVCEKLKQIEGLDQSMLPQYCTTIKKANINGRVLAQCNIDELKKEMN
MNFGDWHLFRSTVLEMRNAESHVVPEDPRFLSESSSGPAPHGEPARRASHNELPHTELSS
QTPYTLNFSFEELNTLGLDEGAPRHSNLSWQSQTRRTPSLSSLNSQDSSIEISKLTDKVQ
AEYRDAYREYIAQMSQLEGGPGSTTISGRSSPHSTYYMGQSSSGGSIHSNLEQEKGKDSE
PKPDDGRKSFLMKRGDVIDYSSSGVSTNDASPLDPITEEDEKSDQSGSKLLPGKKSSERS
SLFQTDLKLKGSGLRYQKLPSDEDESGTEESDNTPLLKDDKDRKAEGKVERVPKSPEHSA
EPIRTFIKAKEYLSDALLDKKDSSDSGVRSSESSPNHSLHNEVADDSQLEKANLIELEDD
SHSGKRGIPHSLSGLQDPIIARMSICSEDKKSPSECSLIASSPEENWPACQKAYNLNRTP
STVTLNNNSAPANRANQNFDEMEGIRETSQVILRPSSSPNPTTIQNENLKSMTHKRSQRS
SYTRLSKDPPELHAAASSESTGFGEERESIL

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Use of the word “we” as a pronoun herein refers generally to laboratory personnel or other contributors who assisted in laboratory procedures and data collection and is not intended to represent an inventorship role by said laboratory personnel or other contributors in any subject matter disclosed herein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, as well as 1.1, 1.2, 1.3, 1.4, 1.5, etc., 2.1, 2.2, 2.3, 2.4, 2.5, etc., and so forth, where the range is not limited solely to integers. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

As noted above, any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of +20% or +10%, or +5%, or +1%, or +0.1% from the specified value, as such variations are appropriate to perform the disclosed methods and as understood by persons having ordinary skill in the art. As used herein, the term “substantially” means that the subsequently described event, circumstance, or sequence completely occurs or that the subsequently described event, circumstance, or sequence occurs to a great extent or degree. For example, the term “substantially” means that the subsequently described event, circumstance, or sequence occurs at least 80% of the time, 90% of the time, or at least 95% of the time, or at least 98% of the time, or comprises at least 80%, at least 90%, at least 95%, or at least 98% of the original.

As used herein any reference to “one embodiment” or “an embodiment” means that a particular element, component, step, feature, structure, or characteristic described in connection with the embodiment is included in at least one embodiment. The appearances of the phrase “in one embodiment” in various places in the specification are not necessarily all referring to the same embodiment.

When two or more active agents described in present disclosure, or their equivalents, are administered, they may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability of an agent to modify the physiological system of an organism without reference to how the agent (“active agent”) has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal, and more particularly, humans. Animals which fall within the scope of the term “subject” as used herein include, but are not limited to, dogs, cats, rats, mice, guinea pigs, chinchillas, horses, goats, ruminants such as cattle, sheep, swine, poultry such as chickens, geese, ducks, and turkeys, zoo animals, Old and New World monkeys, and non-human primates.

“Treatment” refers to therapeutic treatments, such as for promoting wound healing. “Prevention” refers to prophylactic or preventative treatment measures. The term “treating” refers to administering the composition to a patient for therapeutic purposes such as for promoting wound healing.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic effect without excessive adverse side effects (such as toxicity, irritation, and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. The effective amount for a given situation can be determined by one of ordinary skill in the art based on the information provided herein.

The term “ameliorate” means a detectable or measurable improvement in a subject's condition, disease, or symptom thereof. A detectable or measurable improvement includes a subjective or objective decrease, reduction, inhibition, closure, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of completely or partially decreasing, reducing, inhibiting, suppressing, limiting, controlling, or preventing the occurrence, frequency, severity, progression, or duration of a disease or condition, or consequences of the disease or condition.

A decrease or reduction in the worsening of a disease or condition, such as stabilizing the condition or disease, such as a tumor, is also a successful treatment outcome. A therapeutic benefit therefore need not be complete ablation or reversal of the disease or condition, or of any one of, or most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.), such as partial closure of a wound. Effectiveness of a method or use, such as a treatment that provides a potential therapeutic benefit or improvement of a condition or disease, can be ascertained by various methods, measurements, and testing assays.

A sample for testing for the presence of non-benign mutants of KIDINS220 may be obtained from a subject who is being considered as a kidney transplant recipient, or may be obtained from a subject whose biological material, such as a kidney, is intended to be provided for transplantation into a subject. Alternatively, a sample for testing for the presence of non-benign mutants of KIDINS220 may be obtained from a kidney being evaluated for possible transplantation into a subject.

The term “sample” may be a “body fluid sample,” which as used herein is intended to refer to a sample of a bodily fluid obtained from a subject of interest, such as a potential transplant recipient, or a potential transplant donor, or from a tissue or organ of such recipient or donor. In non-limiting embodiments, the body fluid sample may include blood, serum, plasma, cerebrospinal fluid, urine, saliva, sputum, buccal swab, and plural effusions. In certain embodiments, prior to analysis, the body fluid sample may be fractionated or purified, for example by separation of whole blood into serum or plasma components.

Returning now to the detailed description of the various embodiments of the disclosure, KIDINS220 is an integral plasma membrane protein that plays a major role in signaling processes throughout the body. In certain neuroendocrine cells, KIDINS220 acts as a substrate of protein kinase D (PKD), playing a role in the synthesis and secretion of norepinephrine and dopamine. In the nervous system, KIDINS220 functions as a scaffold for neuronal cell membranes, organizes extracellular signaling complexes, and plays an essential role in neuronal cell survival and synaptic plasticity. KIDINS220 also plays a role in the development of the cardiovascular system via its role as a down-stream substrate of vascular endothelial growth factor. Furthermore, within the immune system, the KIDINS220 protein is involved in B-cell and T-cell development and activation.

In accordance with the various cellular signaling pathways in which KIDINS220 plays an important part, mutations within the gene that encodes for this transmembrane protein have been implicated in a variety of human diseases; these include Alzheimer's disease, asthma, cancer, and obesity. KIDINS220 mutations have been linked to obesity via the Spastic paraplegia, Intellectual disability, Nystagmus, and Obesity syndrome (“SINO”). This rare genetic syndrome is associated with cranial and maxillofacial deformities, severe intellectual disability, and obesity, and is caused by heterozygous nonsense KIDINS220 mutations. It has been theorized that the truncated form of KIDINS220 found in patients with SINO results in an inability for adipocytes to differentiate, leading to uncontrolled adipocyte maturation, lipid accumulation, and early-onset obesity despite normal eating and physical activity patterns. Thus, mutations in KIDINS220 have been linked to a genetic mechanism for the development of obesity. SINO remains an incredibly rare genetic syndrome, with only a handful of reported cases worldwide since 2016. However, KIDINS220 mutations in the absence of diagnosed SINO, while still rare, are comparatively more common with a prevalence of approximately 2.3% among over 40,000 individuals with severe obesity.

Obesity, defined as a body mass index (“BMI”) of ≥30 kg/m², is a complex chronic health condition that is associated with an increased risk of several long-term health consequences including cardiovascular disease, hypertension, diabetes, chronic kidney disease (“CKD”), and certain cancers. KIDINS220 is just one of several dozen genes discovered to have strong genetic associations to early-onset (prior to the age of 18) and severe/class III obesity (BMI≥40 kg/m²) since the advent of genome-wide association studies and next-generation sequencing. These genetic discoveries have led to the development of pharmaceuticals with the ability to target gene-specific pathways to treat obesity.

### Genetic Obesity Screening Scale (GOSS)

Traditionally only patients with severe syndromic obesity symptoms such as Prader-Willi Syndrome (PWS) or Bardet-Biedle Syndrome (BBS) were screened for genetic obesity. Over the past decade with the increasing accessibility of genetic testing and the increasing understanding that monogenic obesity can significantly contribute to severe obesity for a larger subset of patients genetic testing has increased. However, due to the high cost of such testing (e.g., $500 to $5000 per test) identifying the appropriate patient population to increase the positivity rate on testing is an important discussion factor for high value care if this is to be implemented nationally on the 100+ million population of people with obesity.

To date there is no widely accepted criteria for when patients should be screened for genetic obesity, particularly in the adult population, which comprises the large majority of both obese (BMI≥30 kg/m²) and severely obese (BMI≥40 kg/m²) individuals. Patients with monogenic obesity may respond differently to both bariatric surgery and standard medical treatment, which makes identifying these patients potentially highly useful. Additionally, there are now FDA approved pharmacotherapies for specific monogenic obesity genes.

Nationally, the most commonly used genetic testing panel is the Uncovering Rare Obesity® program, which is a sponsored screening test for 80 different genetic abnormalities associated with obesity. The national positivity rate for this panel is around 70 to 75%, however this is wildly variable and there is no standardization of when clinicians should order the test.

In our clinic we have tested approximately 1000 patients and have developed a standardized screening workflow to assess if patients should be tested for genetic obesity. This is predominantly utilized in patients ages 15 and above due to the adolescent and adult nature of our clinic.

The assessment is a simple scoring system that is the sum of the patient's BMI and their score on the Gormley Binge Eating Scale (BES). The BES has a maximum score of 46. When the patient's self-reported score is added to the BMI, a resulting number of 70 or greater indicates automatic genetic testing. In the past we had tested patients without this and had a higher negative result rate. The case of use of this score is that it requires a single patient self-reported study and a vital sign and can be performed in a standardized fashion on intake without requiring extensive clinician history. This provides a significant advancement in the testing of adult patients for monogenic obesity compared to the current standard of care which does not exist.

Mirroring worldwide trends, obesity rates are increasing among individuals with CKD and ESRD. Obesity plays a prominent, if complex role in these patients: obesity alone (in addition to its associated metabolic syndromes such as hypertension and diabetes) can not only cause CKD but can also hasten the progression of CKD. As CKD progresses to ESRD, kidney transplantation offers a longer survival benefit and better quality of life than hemodialysis or other renal replacement therapies. However, those with ESRD and obesity face unique challenges when planning for organ transplantation, as obesity is associated with an increased risk of complications both during and after surgery. Such complications include, but are not limited to, perioperative infection, delayed graft function, and development of complications from long-term immunosuppressive medications. As a result of the increased intra- and post-operative risks and in the absence of published international guidelines regarding transplant in the setting of severe obesity, many transplantation centers have developed BMI “cutoffs” that patients must meet in order to be eligible for transplantation. For this reason, patients with ESRD and obesity are often required to achieve significant weight loss before they may qualify for a kidney transplant.

Despite the growing evidence that mutations in KIDINS220 play a role in genetic neurologic conditions and as genetic obesity syndromes, prior to the present work, there have been no studies linking KIDINS220 or its mutations to chronic renal disease or ESRD. The present disclosure identified a cohort of patients who were unrelated and had various attributable causes to their renal disease, but who were diagnosed with both ESRD and severe obesity and who have a significantly higher rate of KIDINS220 mutations compared to genetically tested patients with severe obesity nationwide.

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures and methods.

### Methods

The present work utilized retrospective chart review to identify individuals with obesity (BMI≥30 kg/m²) and ESRD (defined as a glomerular filtration rate (“GFR”) below 15 mL/min) who presented to an interdisciplinary obesity medicine and bariatric surgery clinic for pre-kidney transplant weight management therapy. Twenty-one individuals met these criteria. Twenty of the 21 reported early-onset severe obesity (BMI≥40 kg/m² with a history of childhood obesity) concerning for a genetic cause, and were therefore screened for genes associated with genetic obesity syndromes. Screening was performed by utilizing the mail-in Uncovering Rare Obesity® genetic testing panel that analyzes buccal swab samples for polymorphisms in 79 genes with known obesogenic variants.

Results of genetic testing in these 20 patients were compared to the national positivity rates of identified obesogenic polymorphisms.

### Results

Twenty patients with both obesity and ESRD met criteria and underwent testing for genetic causes of obesity, 14 (70%) of whom were male. Fifteen (75%) of the 20 tested individuals were found to possess at least one obesogenic polymorphism. Those who possessed obesogenic polymorphisms were also predominantly (12/15, 80%) male.

Four (20%) of the 20 tested individuals were found to have a polymorphism within the KIDINS220 gene; again, reflecting the demographics of the tested sample, these individuals were predominantly (3/4, 75%) male.

Utilizing a chi-squared analysis, the rate of identified KIDINS220 polymorphisms in the present cohort of 20 patients diagnosed with both ESRD and obesity was compared to the reported national hit rates within the KIDINS220 gene among all tested individuals with obesity. When compared to the national KIDINS220 polymorphism hit rate (approximately 2.3%), the present sample of individuals with both obesity and ESRD had a significantly higher positivity rate of KIDINS220 polymorphisms at 20% (χ²=27.8, P<0.0001).

Presented below is a case series of the four patients with both early onset obesity and ESRD who were found to have polymorphisms within the KIDINS220 gene.

### Case Presentations

### Case 1:

A 43-year-old Caucasian male presented in August 2021, for medical weight management in preparation for renal transplant. Medical history was significant for binge eating disorder, prior methamphetamine and tobacco use, obstructive sleep apnea, and dyslipidemia. Decreases in this patient's renal function were first noted in February 2020, during routine laboratory evaluation. He was hospitalized in October 2020, with an acute kidney injury; at that time, testing demonstrated significant proteinuria (urine protein to creatinine ratio of 2,990 mg/g; normal <200 mg/g) and renal biopsy would reveal focal segmental glomerulosclerosis. Hemodialysis was initiated five months later, in March 2021. At presentation for weight management prior to renal transplant, the patient weighed 178.8 kg (BMI 46.7 kg/m²). He reported he had already lost 58.97 kg prior to presentation. Medical management for weight loss was initiated, and patient would later undergo sleeve gastrectomy in August 2022. Medical weight loss therapy was continued postoperatively, until patient was ultimately cleared for renal transplant in October 2023, at 117.48 kg (BMI 30.7 kg/m²). Genetic testing revealed mutations within KIDINS220 and PPARG.

### Case 2:

A 53-year-old African American male presented in December 2021, for medical weight management to qualify for renal transplant. Medical history was significant for coronary artery disease, atrial fibrillation, hypertension, stroke, and history of tobacco use. His renal dysfunction began at age 14 and was of unknown cause. Hemodialysis was initiated in 2013 at the age of 43. At his initial weight management appointment, he weighed 126.8 kg (BMI 41.3 kg/m²). He was started on medical therapy for weight loss and would ultimately undergo sleeve gastrectomy in June 2022. He resumed medical weight loss therapy after surgery due to early postoperative weight plateau and regain. As of this filing, the patient had achieved a weight of 102.0 kg (BMI 33.2 kg/m²). He underwent genetic screening and was found to have a mutation in KIDINS220. The patient is currently listed for renal transplant.

### Case 3:

A 36-year-old Caucasian male presented in May 2022, for evaluation for medical weight management prior to renal transplant. Medical history was significant for systemic lupus erythematosus, type 2 diabetes mellitus, coronary artery disease, hypertension, hyperlipidemia, and tobacco use. He was initially diagnosed with chronic kidney disease at the age of 28, with significant proteinuria (urine protein to creatinine ratio of 2,588 mg/g; normal <200 mg/g) and biopsy-proven focal segmental glomerulosclerosis. At his initial appointment, the patient weighed 160.5 kg (BMI 44.3 kg/m²). He achieved 25% total body weight loss through medical management that involved both oral and injectable anti-obesity medications, for a final weight of 120.4 kg (BMI 33.2 kg/m²). On genetic testing, the patient was found to have polymorphisms within three obesogenic genes: KIDINS220, PLXNA3, and PCSK1.

### Case 4:

A 62-year-old Caucasian female presented in August 2022, for medical weight management to qualify for renal transplant. Medical history was significant for hypertension, obstructive sleep apnea, gastroesophageal reflux disease, type 2 diabetes mellitus, Bardet-Biedl syndrome, and hyperlipidemia. Her kidney dysfunction was first noted in March 2018, during hospitalization for acute kidney injury. During the progression of her renal disease, she had multiple hospitalizations for urinary tract infection, bacteremia, and sepsis. At her initial appointment, she weighed 165.3 kg (BMI 60.6 kg/m²) and began medical therapy for weight loss with injectable anti-obesity medication. Her lowest weight achieved to date is 147.9 kg (BMI 54.3 kg/m²). Upon genetic testing, she was found to have mutations in KIDINS220 and IFT172.

### Discussion

The significantly higher mutant KIDINS220 positivity rate in this sample of patients with both obesity and ESRD (25%), compared to the positivity rate of all individuals with obesity who were tested for genetic causes of obesity (2.3%, p<0.01) indicates that the present sample is inherently different than the general population of individuals with obesity. It was therefore postulated that mutations within KIDINS220 may play a modulatory role in the progression of chronic kidney disease by predisposing patients to obesity.

Obesity contributes to the development and progression of CKD, in part by predisposing patients to hypertension and type 2 diabetes. However, obesity is also a strong independent risk factor for CKD even in the absence of these comorbidities. The mechanism by which obesity can directly cause CKD is unclear. One leading theory is based upon the known changes that occur within the nephron as a response to increased adiposity, namely a compensatory increase in GFR, increased renal plasma flow, increased sodium retention, and activation of the renin-angiotensin-aldosterone system. Over time, this hyperfiltration may cause increased pressure within the glomerulus and eventual glomerulopathy, leading to chronic kidney disease.

Obesity-related hyperfiltration has been linked to CKD via Obesity-Related Glomerulopathy (“ORG”), a secondary form of focal-segmental glomerulosclerosis (FSGS) that occurs in individuals with a BMI≥30 kg/m². ORG is characterized by near nephrotic-range proteinuria in the absence of a nephrotic syndrome, glomerulomegaly, and mild podocyte depletion. Two of the 4 individuals with obesity presented here had CKD attributable to biopsy-proven FSGS, which is consistent with the presently identified connection between KIDINS220, obesity, and the development of CKD.

Because obesity plays a prominent role in the development and progression of CKD, the relationship between obesity and CKD raises unique management challenges in individuals hoping to pursue renal transplant. A genetic target that links both obesity and kidney disease would therefore be valuable for early identification and intervention to prevent progression to ESRD.

Obesity and ESRD result in a substantial reduction in the quality of life of those they affect and place a significant financial burden on both patients and the healthcare system. Establishment of a relationship between KIDINS220, ESRD, and obesity will result in widespread benefits by enabling early intervention and prevention of the progression of CKD to ESRD.

In summary, in at least one non-limiting embodiment, the present disclosure is directed to a method of evaluating a subject for a renal condition (e.g., ESRD), wherein the subject is either obese or severely obese, and treating the subject based on the evaluation, wherein the method comprises (a) performing an assay configured to detect a non-benign Kinase D-interacting substrate of 220 kDA (KIDINS220) variant protein in a body fluid sample obtained from the subject, (b) determining that the subject has an elevated risk for end-stage renal disease (ESRD) when one or more non-benign KIDINS220 variants are present in the body fluid sample, and (c) treating the subject having the elevated risk for ESRD with a compatible treatment regimen, wherein the compatible treatment regimen comprises one or more of (1) initiating renal replacement therapy, (2) withdrawing delivery of compounds that are known to be damaging to the kidney, (3) delaying procedures that are known to be damaging to the kidney, (4) modifying diuretic administration, (5) monitoring and optimizing hemodynamics and fluid administration, and (6) administration of a weight loss pharmacotherapy selected from an appetite suppressant and a GLP-1 receptor agonist. The GLP-1 receptor agonists which may be used include but are not limited to semaglutide (OZEMPIC, RYBELSUS, WEGOVY), liraglutide (VICTOZA, SAXENDA), dulaglutide (TRULICITY), exenatide (BYETTA), tirzepatide (MOUNJARO), albiglutide (TANZEUM), lixisenatide (LIXUMIA, ADLYXIN), and loxenatide (FU LEIMAI). Appetite suppressants which may be used include but are not limited to orlistat, phentermine, topiramate, phentermine-topiramate, lorcaserin, diethylpropion, bupropion, fluoxetine, sertraline, naltrexone, bupropion-naltrexone, miglitol, acarbose, desipramine, zonisamide, rimonabant, metformin, phendimetrazine, and benzphetamine.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of evaluating a subject for End Stage Renal Disease (ESRD), wherein the subject is obese or severely obese, and treating the subject based on the evaluation, the method comprising:
performing an assay configured to detect a non-benign Kinase D-interacting substrate of 220 kDA (KIDINS220) variant protein in a body fluid sample obtained from the subject;
determining that the subject has an elevated risk for ESRD when one or more non-benign KIDINS220 variants are present in the body fluid sample; and
treating the subject having the elevated risk for ESRD with a compatible treatment regimen, wherein the compatible kidney treatment regimen comprises one or more of (1) initiating renal replacement therapy, (2) withdrawing delivery of compounds that are known to be damaging to the kidney, (3) delaying procedures that are known to be damaging to the kidney, (4) modifying diuretic administration, (5) monitoring and optimizing hemodynamics and fluid administration associated with the kidney, (6) administering a weight loss pharmacotherapy comprising a GLP-1 receptor agonist, and (7) administering a weight loss pharmacotherapy comprising an appetite suppressant.

2. The method of claim 1, wherein the GLP-1 receptor agonist is selected from semaglutide, liraglutide, dulaglutide, exenatide, tirzepatide, albiglutide, lixisenatide, and loxenatide.

3. The method of claim 1, wherein the appetite suppressant is selected from orlistat, phentermine, topiramate, phentermine-topiramate, lorcaserin, diethylpropion, bupropion, fluoxetine, sertraline, naltrexone, bupropion-naltrexone, miglitol, acarbose, desipramine, zonisamide, rimonabant, metformin, phendimetrazine, and benzphetamine.
